(19) United States
(12) Patent Application Publication
(10) Pub. No.: US 2009/0202510 A1
(43) Pub. Date: Aug. 13, 2009

(54) ALTERED SUMOYLATION OF LAMIN A PROTEIN ASSOCIATED WITH DILATED CARDIOMYOPATHY

(75) Inventor: Kevin D. SARGE, Lexington, KY (US)

Correspondence Address: CROWELL & MORING LLP;INTELLECTUAL PROPERTY GROUP, P.O. BOX 14300, WASHINGTON, DC, 20044-4300, US

(73) Assignee: University of Kentucky Research Foundation, Lexington, KY (US)

(21) Appl. No.: 12/357,849

(22) Filed: Jan. 22, 2009

Related U.S. Application Data
(60) Provisional application No. 61/006,607, filed on Jan. 23, 2008.

(51) Int. Cl. A61K 38/53; C12Q 1/02; G01N 33/68; G01N 33/536; G01N 33/543; A61K 31/7088

(52) U.S. Cl. 424/94.5; 435/29; 436/86; 436/536; 436/518

(57) ABSTRACT

This invention relates to methods of diagnosing dilated cardiomyopathy using analysis of the lamin A protein and sumylation of same, as well as methods for treating dilated cardiomyopathy, comprising enhancing the sumoylation of the lamin A protein in a subject in need thereof.

### TECHNICAL FIELD

This invention relates to methods of diagnosing dilated cardiomyopathy using analysis of the lamin A protein and sumylation of same, as well as methods for treating dilated cardiomyopathy, comprising enhancing the sumoylation of the lamin A protein in a subject in need thereof.

### STATEMENT AS TO RIGHTS TO INVENTIONS MADE UNDER FEDERALLY SPONSORED RESEARCH OR DEVELOPMENT

This research was supported by Grant No. GM64606 from the National Institutes of Health (NIH). The Government has certain rights in the invention.

### BACKGROUND OF THE INVENTION

The lamin A protein plays an important role in the structure and function of the nucleus, and mutations in the lamin A gene cause a large number of different human diseases, including cardiomyopathies, muscular dystrophies, and Hutchinson-Gilford Progeria Syndrome (1-7). Post-translational attachment of Small Ubiquitin-like Modifier (SUMO) proteins to lysine residues in target proteins, or sumoylation, is an important regulator of protein functional properties (8-14). SUMO proteins are covalently attached to target lysine residues by the SUMO E2 enzyme, ubc9, and these substrate lysines are typically found within the consensus sequence ΨKXE (Ψ represents hydrophobic amino acids) (15-18). Cells express three major SUMO paralogs, SUMO-1, SUMO-2, and SUMO-3, with SUMO-2/SUMO-3 being much more similar to each other than to SUMO-1 (8-14).

### SUMMARY

The present inventors have found that the lamin A protein is sumoylated, and that a decrease in sumoylation of lamin A is correlated with dilated cardiomyopathy. The present invention provides a method of diagnosing dilated cardiomyopathy in a subject, comprising (a) obtaining a biological test sample from said subject, wherein said biological test sample comprises lamin A protein; (b) measuring the sumoylation of said lamin A protein from said biological test sample; and (c) determining whether said lamin A protein from said biological test sample has decreased sumoylation by comparing the sumoylation obtained in step (b) with a standard sumoylation level; wherein decreased sumoylation of said lamin A protein from said biological test sample relative to said standard sumoylation level is indicative of dilated cardiomyopathy. In another embodiment, the present invention also provides a method for treating dilated cardiomyopathy, comprising enhancing the sumoylation of the lamin A protein in a subject in need thereof.

### BRIEF DESCRIPTION OF THE DRAWINGS

FIG. 1. Lamin A is sumoylated at lysine 201 by SUMO-2. FIG. 1A is a schematic showing location of a match (MKEE) to the sumoylation site consensus sequence (ΨKXE) surrounding lysine 201 in the rod-containing domain of lamin A. FIG. 1B is an immunoprecipitation analysis of sumoylation of wildtype and K201R lamin A by SUMO-1 or SUMO-2. Extracts of HeLa cells transfected with GFP-fusion constructs of wildtype lamin A or K201R mutant lamin A and HA-tagged SUMO-1 or SUMO-2 plasmids were subjected to immunoprecipitation using anti-GFP antibodies followed by anti-HA Western blot. The levels of the transfected GFP-Lamin A proteins in the cell lysates were determined by Western blot assay using GFP antibody.

FIG. 2. Non-sumoylatable K201R mutant lamin A exhibits an altered subcellular localization pattern. Wildtype or K201R lamin A GFP-fusion expression plasmids were transfected into HeLa cells along with the HA-SUMO-2 expression plasmid, and the subcellular localization of the GFP-lamin proteins examined by fluorescence microscopy using the green channel. DNA was visualized by staining with Hoechst 33342.

FIG. 3. E203G and E203K mutant lamin A proteins exhibit decreased sumoylation. FIG. 3A is a schematic showing the location of the E203G and E203K mutations in lamin A associated with familial dilated cardiomyopathy at the conserved glutamic acid residue of the sumoylation site consensus sequence (ΨKXE) surrounding lysine 201. FIG. 3B shows extracts of HeLa cells transfected with GFP-fusion constructs of wildtype lamin A, E203G mutant lamin A, or E203K mutant lamin A along with HA-tagged SUMO-2 were subjected to immunoprecipitation with anti-GFP antibodies followed by anti-HA Western blot. The levels of transfected GFP-Lamin A proteins in the cell lysates were determined by Western blot assay using GFP antibody.

FIG. 4. E203G and E203K mutant lamin A proteins exhibit altered subcellular localization patterns. GFP fusion constructs of wildtype lamin, or the mutant lamin A proteins associated with familial dilated cardiomyopathy, E203G and E203K, were transfected into HeLa cells along with HA-SUMO-2 expression plasmid, and then the subcellular localization of these proteins was examined by fluorescence microscopy using the green channel. DNA was visualized by staining with Hoechst 33342.

FIG. 5. Protein sequence of normal human lamin A. The sequence of human lamin A protein (SEQ ID NO: 1) is known, and a sample comprising normal (wildtype) human lamin A has been deposited under GenBank Accession Number P02545.

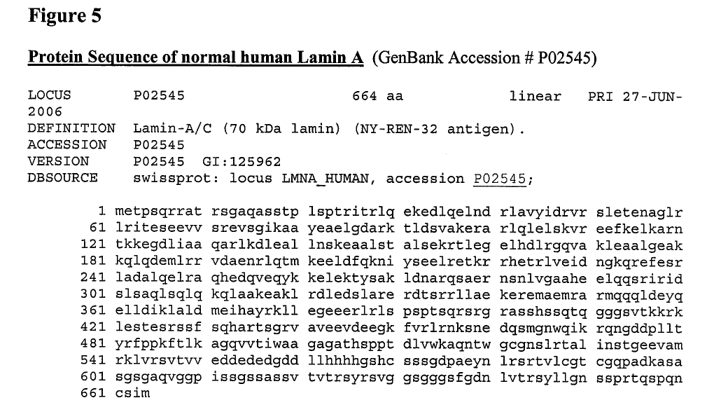

### DETAILED DESCRIPTION OF THE INVENTION

The present invention provides a diagnostic assay for diagnosing dilated cardiomyopathy, comprising measuring the sumoylation of lamin A protein in a biological sample obtained from a subject and comparing the sumoylation with a standard sumoylation level, whereby a decrease in sumoylation compared to the standard sumoylation level is indicative of dilated cardiomyopathy.

According to the present invention, a decreased level of lamin A sumoylation in a biological sample from a subject may indicate a predisposition for the development of dilated cardiomyopathy, or may provide a means for detecting the disease prior to the appearance of actual clinical symptoms. Diagnosis according to the present invention may thus allow health professionals to employ preventative measures or aggressive treatment earlier, thereby preventing the development or further progression of the disease. In addition, the diagnostic test according to the present invention may enable diagnosis of dilated cardiomyopathy related to defective lamin A sumoylation in a subject already suffering from a previously uncharacterized form of dilated cardiomyopathy. By “uncharacterized form of dilated cardiomyopathy” is meant a form of dilated cardiomyopathy that may or may not be related to decreased sumoylation of lamin A protein.

The present invention is directed to methods for diagnosing dilated cardiomyopathies that relate to a decreased sumoylation of lamin A protein. The dilated cardiomyopathy may be a familial dilated cardiomyopathy. The decreased lamin A sumoylation can be related to one or more mutations in the lamin A protein. The mutations can be substitution, deletion, or addition mutations. In one embodiment, the mutation in the lamin A protein disrupts the lamin A sumoylation consensus sequence. In another embodiment, the mutation comprises a substitution, deletion, or addition mutation within the consensus sequence. For example, the mutation can comprise substitution of the glutamic acid residue at amino acid position 203 of SEQ ID NO: 1. The glutamic acid residue can be substituted with a glycine or a lysine residue. In another embodiment, the mutation is in a sequence of the lamin A protein that is important for binding to one or more SUMO E3 proteins, which function to enhance sumoylation of proteins by interacting with both the SUMO E2 enzyme (ubc9) and the sumoylation substrate protein, thereby helping bring them together.

Alternatively, decreased sumoylation of lamin A may be unrelated to any mutation in the lamin A protein. For example, decreased sumoylation of lamin A can be caused by a defect in the enzymatic pathway that is responsible for attachment of the SUMO protein to lamin A, such as a mutation in and/or a decreased level of expression of the SUMO E2 protein.

In the present invention, the term “measuring the sumoylation of lamin A protein” refers to qualitatively or quantitatively measuring or estimating the level of sumoylation of lamin A from a biological test sample either directly (e.g., by determining or estimating absolute level of sumoylation) or relatively (e.g., by comparing to the lamin A sumoylation level in a biological control sample). In one embodiment, the lamin A sumoylation level in the biological test sample is measured or estimated and compared to a standard lamin A sumoylation level, the standard being taken from a biological control sample obtained from an individual not having dilated cardiomyopathy or being determined by averaging levels from a population of individuals not having the disease. As will be appreciated in the art, once a standard lamin A sumoylation level is known, it can be used repeatedly as a standard for comparison.

Measurement of lamin A sumoylation can be carried out in any suitable biological test sample obtained from a subject. “Biological test sample” means any biological sample obtained from a subject which contains lamin A protein. Suitable sources of biological test sample include body fluids (such as the following non-limiting examples: blood, blood products, serum, saliva, sputum, amniotic fluid, lymph, urine, breast milk, secretions, interstitial fluid, and spinal fluid) or other tissue sources found to contain lamin A. In one embodiment, the biological test sample is a tissue biopsy. In another embodiment, the biological test sample is a cell extract. In yet another embodiment, the biological test sample is a lymphocyte extract. Methods for obtaining suitable biological test samples are well known in the art.

As used herein, the term “decreased sumoylation” refers to any measurable decrease as compared to a standard sumoylation level, wherein the decrease correlates to dilated cardiomyopathy. In certain embodiments, decreased sumoylation means that the lamin A protein from the biological test sample exhibits from about 5% to about 100% less sumoylation as compared to the standard sumoylation level, such as about 10%, 20%, 30%, 40%, 50%, 60%, 70%, 80%, or 90% less than the standard. In one embodiment, the lamin A protein from the biological test sample exhibits about 50% or less than 50% sumoylation as compared to the standard. In another embodiment, the lamin A protein from the biological test sample exhibits about 80% or less than 80% sumoylation as compared to the standard. In yet another embodiment, the lamin A protein from the biological test sample exhibits no detectable sumoylation.

The sumoylation of lamin A protein can be determined using any number of techniques known in the art. For example, lamin A sumoylation can be measured using an antibody that specifically recognizes the sumoylated form of lamin A (but not the non-sumoylated form of lamin A), or vice versa. In one embodiment, such antibodies can be used in Western blot assays comparing lamin A from a test sample with a lamin A standard or control to look for the presence, absence, and/or intensity of a band corresponding to the size of sumoylated lamin A (approximately 150 kDa in size on SDS-PAGE according to the present invention). In another example, lamin A protein from a biological test sample can be immunoprecipitated using lamin A antibodies that recognize both the sumoylated and non-sumoyled forms of lamin A, followed by Western blotting using anti-SUMO-2 antibodies.

Suitable subjects for the present invention include, for example, subjects suspected of having dilated cardiomyopathy or being predisposed to having dilated cardiomyopathy. A suitable subject may also be a subject suffering from an uncharacterized form of dilated cardiomyopathy. In one embodiment, the subject is a mammal, such as a dog, a cat, a cow, a horse, or a human. In another embodiment, the subject is a human.

The present invention also provides a method for treating dilated cardiomyopathy, comprising enhancing or increasing the sumoylation of lamin A protein in a subject in need thereof.

“Treatment” or “treating,” as used herein, refers to complete elimination as well as to any clinically or quantitatively measurable reduction in the condition for which the subject is being treated. The treatment methods of the present invention involve treating dilated cardiomyopathy by “enhancing” or “increasing” the sumoylation of lamin A protein. “Enhancing” or “increasing,” as used herein, refers to enhancing or increasing the sumoylation of lamin A protein in a subject by an amount sufficient for treating the dilated cardiomyopathy.

A “subject in need thereof” refers to any subject who could benefit from the inventive method of treatment. In certain embodiments, a subject in need thereof is a subject predisposed for the development of dilated cardiomyopathy, a subject having dilated cardiomyopathy but not exhibiting any clinical symptoms, or a subject having dilated cardiomyopathy and suffering from the symptoms of dilated cardiomyopathy. The dilated cardiomyopathy is related to decreased sumoylation of lamin A protein. The subject in need thereof may be a mammal, such as a dog, a cat, a cow, a horse, or a human. In one embodiment, the subject is a human.

The sumoylation of lamin A can be enhanced using any means known in the art. For example, the inventive method for treating dilated cardiomyopathy can comprise administering a virus (such as an adeno-associated virus) that can deliver to the subject's cells an expression construct comprising either the SUMO E2 enzyme (ubc9) or a SUMO E3 protein that functions to enhance sumoylation of lamin A by helping ubc9 bind to the lamin protein. SUMO E3 proteins enhance sumoylation by interacting with both the substrate and the ubc9, thereby helping bring the ubc9 and substrate together.

### EXAMPLES

### Experimental Procedures

Cell culture and plasmids—HeLa cells were cultured in DMEM medium (CELLGRO®) supplemented with 10% FBS and 1× antibiotic-antimycotic (Gibco, 100×) in 5% CO₂. Transfection was performed using EFFECTENE® transfection reagent (Qiagen), following the manufacturer's protocol. Immunoprecipitation analysis and fluorescence microscopy were performed 48 hrs after the transfection. The GFP-lamin A plasmid was constructed from the pcDNA3.1 -LMNA plasmid (which was a generous gift of Dr. Gibson Zhong). The coding region of the lamin A protein was cut out with EcoR I and BamHI digestion, and ligated into pEGFP-C2 vector (Clontech). Mutagenesis PCR was performed to generate GFP-Lamin A K201R, E203G, and E203K mutants. The primers used for the mutagenesis were as follows (only top primers are listed, bottom primers are the reverse complements of each of these): 5′-CTG CAG ACC ATG AGG GAG GAA CTG GAC-3′ (SEQ ID NO: 2)for K201R; 5′-ACC ATG AAG GAG GGA CTG GAC TTC CAG-3′ (SEQ ID NO: 3) for E203G; and 5′-ACC ATG AAG GAG AAA CTG GAC TTC CAG-3′ (SEQ ID NO: 4) for E203K. HA-SUMO-1 and HA-SUMO-2 were expressed using pcDNA3-HA-SUMO-1 and pcDNA3-HA-SUMO-2 plasmids (kindly provided to us by Dr. Kim Orth).

Immunoprecipitation analysis—For each immunoprecipitation, 2 plates of transfected cells were collected, and the cell pellet was re-suspended in 500 μl RIPA buffer (150 mM NaCl, 50 mM Tris-HCl pH 7.4, 1% NP-40, 0.2% SDS, 0.25% sodium deoxycholate, 1 mM EDTA), with 1× protease inhibitor cocktail (Roche), 1 mM DTT, 20 mM N-ethylmaleimide (added fresh). Cell lysis was performed by sonication after which the sample was incubated on ice for 20 minutes. After centrifugation at 10,000 rpm, 4° C. for 10 minutes, the supernatant was transferred to a fresh tube, and 20 μl of the whole cell lysate was removed for analysis of the level of transfected GFP-Lamin A. 300 μl of 50% Protein G Sepharose slurry (Amersham Biosciences) was washed 3 times with PBS, and re-suspended in RIPA buffer to make a 50% slurry. The cell lysate was precleared by mixing it with 150 μl of this slurry and 10 μg goat IgG, and incubating at 4° C. for 60 minutes. After centrifugation at 4000 rpm, 4° C. for 1 minute, the supernatant was transferred to a fresh tube, and mixed with 10 μg anti-GFP antibody (Bethyl Inc.). After incubation at 4° C. for 60 minutes, 150 μl of 50% Protein G Sepharose slurry was added and incubated at 4° C. for 60 minutes. The beads were washed with RIPA buffer 4 times, and then boiled in 50 μl 4× SDS-PAGE loading buffer. After brief centrifugation, the supernatant was subjected to SDS-PAGE and Western blot using anti-HA antibody.

Western blot and antibodies—SDS-PAGE and Western blot were performed following standard procedures. The antibodies and dilutions used to probe the Western blots were as follows. Goat anti-GFP antibody (Bethyl) was used at 1:2000, and mouse anti-HA antibody (gift from Dr. Douglas Andres lab) was used at a dilution of 1:2000.

Fluorescence microscopy—HeLa cells were seeded on coverslips. At 48 hours after transfection with the wildtype and point mutant GFP-lamin A expression constructs, Hoechst 33342 and verapamil were added to the medium to final concentrations of 5 μg/ml and 50 μg/ml, respectively. After incubation at 37° C. for 30 minutes, the coverslips were washed twice with PBS, and then incubated in 3.7% paraformaldehyde at room temperature for 20 minutes. After two washes with PBS and a brief wash with distilled water, the coverslips were wicked on a KIMWIPE® to partially dry, and then mounted onto a slide spotted with 15 82 VECTORSHIELD™ (Vector Laboratories). Excess fluid was wicked from the coverslips, and the edges of the coverslip sealed with fingernail polish. The fluorescence was then visualized using a Nikon fluorescent microscope, and pictures taken with a Nikon SPOTCAM digital-imaging camera.

### Results and Discussion

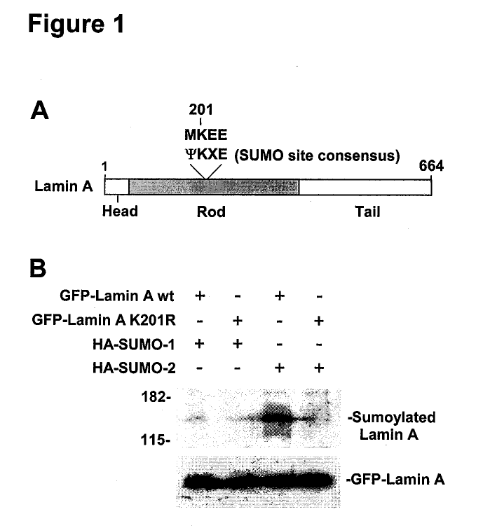

Analysis of the lamin A amino acid sequence revealed a match to the sumoylation consensus sequence ΨKXE (MKEE) surrounding lysine 201 in the rod-containing domain of lamin A (FIG. 1A). To test whether the lamin A protein is sumoylated, and if so, whether the modification is occurring at lysine 201, HeLa cells were transfected with mammalian expression plasmids encoding GFP-fusion constructs of wildtype lamin A and lamin A in which lysine 201 was changed to a non-sumoylatable arginine (K201R), along with expression constructs encoding HA-tagged SUMO-1 or SUMO-2. Extracts of the transfected cells were subjected to immunoprecipitation with anti-GFP antibodies followed by anti-HA Western blot. The results of this experiment, shown in FIG. 1B, suggest that the wildtype lamin A protein is covalently modified by SUMO-2, but not as efficiently sumoylated by SUMO-1. The results also show that the modification by SUMO-2 is not observed for the K201R lamin A mutant protein, suggesting that lysine 201 is the site of SUMO-2 attachment in this protein.

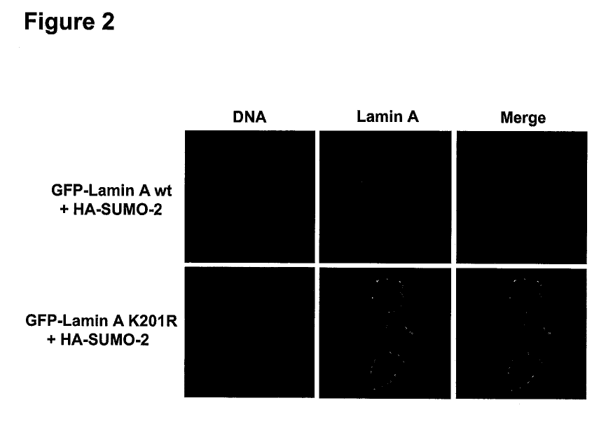

Sumoylation plays an important role in regulating the functional properties of target proteins in cells (8-14). The wildtype lamin A protein exhibits a characteristic pattern of localization at the nuclear periphery (1-7). Based on these findings, it was hypothesized that sumoylation of lamin A at lysine 201 may be important for this localization pattern. To test this hypothesis, HeLa cells were transfected with the wildtype or K201R lamin A GFP-fusion expression plasmids along with the HA-SUMO-2 expression plasmid, and then examined by fluorescence microscopy. As shown in FIG. 2, the wildtype lamin A GFP-fusion protein exhibits the typical pattern of relatively continuous nuclear peripheral localization. However, the K201R lamin A GFP-fusion protein shows an altered localization pattern, with the mutant protein appearing to concentrate into foci. These results suggest that sumoylation of lamin A is important for the normal pattern of subcellular localization of this protein.

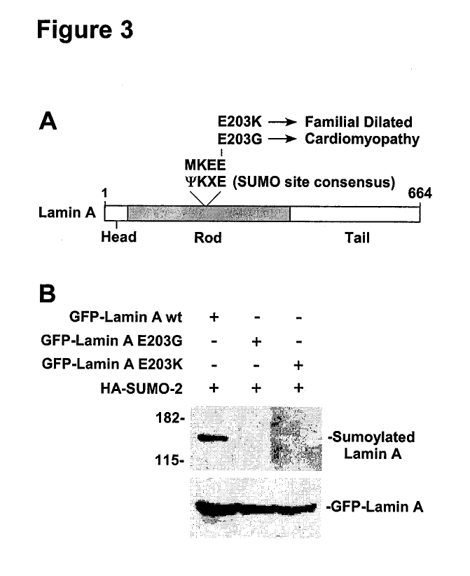

The glutamic acid residue at the fourth position in the sumoylation consensus sequence ΨKXE is known to be important for the efficiency of SUMO addition to the nearby lysine in this sequence (17, 18). Relevant to this, two different mutations of the human lamin A gene have been identified which change the glutamic acid at this position (E203) in the sumoylation consensus sequence of this protein to a different amino acid (FIG. 3A) (20, 21). In one of these mutants glutamic acid 203 is changed to glycine (E203G), while in the other it is changed to lysine (E203K). Both the E203G and E203K mutations of lamin A are associated with familial dilated cardiomyopathy and conduction system disease (20, 21), but the underlying mechanism by which these mutations alter lamin A function and lead to these diseases is not known. Based on the results demonstrating that sumoylation of the lamin A protein occurs at lysine 201, and on previous results indicating the importance of glutamic acid for sumoylation at the preceding lysine of the ΨKXE sumoylation consensus sequence (17, 18), it was hypothesized that the E203G and E203K mutations could mediate their deleterious effects by resulting in decreased sumoylation of the lamin A protein. To test the feasibility of this hypothesis, a transfection-immunoprecipitation experiment similar to that shown above in FIG. 1 was performed, except that the sumoylation of wildtype GFP-lamin A was compared to that of GFP-lamin A constructs containing the E203G or E203K mutations. The results of this experiment, shown in FIG. 3B, indicate that both the E203G and E203K mutant lamin A proteins exhibit decreased sumoylation compared to the wildtype lamin A protein. These results demonstrate that the cardiomyopathy-causing E203G and E203K lamin A mutants are associated with loss of sumoylation of the lamin A protein, supporting the hypothesis that defective lamin A sumoylation could be the underlying molecular defect that leads to this laminopathy.

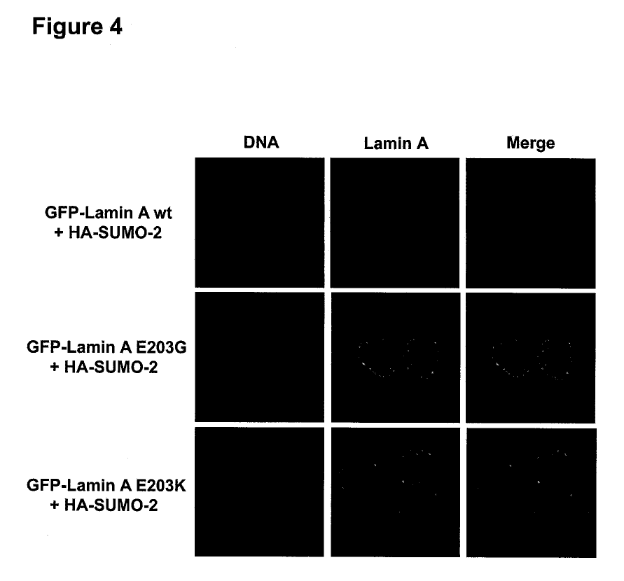

Analysis of the subcellular localization of GFP fusion constructs of the E203G or E203K mutant lamin A proteins by fluorescence microscopy revealed that both of these mutant proteins exhibit altered localization patterns similar to that of the K201 R mutant lamin A, in which these proteins are concentrated in foci, in contrast to the more continuous appearance of the wildtype lamin A at the nuclear periphery (FIG. 4). In light of the shared defect in sumoylation of the K201R, E203G, and E203K mutant lamin A proteins, the similarity between the subcellular localization patterns of the E203G/E203K mutant lamin A proteins shown in this figure and that of the K201R mutant lamin A protein (shown above in FIG. 2) suggests that the altered localization patterns of all three of these mutant lamin A proteins vs. wildtype lamin A are due to their decreased sumoylation.

The results of these experiments indicate that lysine 201 of lamin A is a target of covalent modification by the SUMO-2 protein; that this sumoylation is important for the normal pattern of subcellular localization of the lamin A protein; and that lamin A sumoylation is decreased in the mutant E203G and E203K lamin A proteins that cause familial dilated cardiomyopathies. Finally, the results also indicate that the mutant E203G and E203K lamin A proteins exhibit altered subcellular localization patterns that are very similar to that of the SUMO attachment site mutant K201R lamin A protein, suggesting that altered sumoylation of lamin A is the underlying molecular mechanism of familial dilated cardiomyopathies associated with the E203G and E203K lamin A mutations. This study provides the first example of a human disease-causing mutation occurring in a crucial residue of a sumoylation consensus sequence and resulting in altered sumoylation of the mutant protein.

The previous examples are provided to illustrate but not to limit the scope of the claimed inventions. Other variants of the inventions are encompassed by the appended claims and will be readily apparent to those of ordinary skill in the art.

All publications, patents, patent applications and other references cited herein are hereby incorporated by reference.

### REFERENCES

1. Capell, B. C., and Collins, F. S. (2006) Nat. Rev. Genet. 7, 940-952.
2. Parnaik, V. K., and Manju, K. (2006) J. Biosci. 31, 405-421.
3. Broers, J. L., Ramaekers, F. C., Bonne, G., Yaou, R. B., and Hutchison, C. J. (2006) Physiol. Rev. 86, 967-1008.
4. Mattout, A., Dechat, T., Adam, S. A., Goldman, R. D., and Gruenbaum, Y. (2006) Curr. Opin. Cell Biol. 18, 335-341.
5. Smith, E. D., Kudlow, B. A., Frock, R. L., and Kennedy, B. K. (2005) Mech. Ageing Dev. 126, 447-460.
6. Gruenbaum, Y., Margalit, A., Goldman, R. D., Shumaker, D. K., and Wilson, K. L. (2005) Nat. Rev. Mol. Cell. Biol. 6, 21-31.
7. Verstraeten, V. L., Broers, J. L., Ramaekers, F. C., and van Steensel, M. A. (2007) Curr Med. Chem. 14, 1231-1248.
8. Dohmen, R. J. (2004) Biochim. Biophys. Acta 1695, 113-131.
9. Johnson, E. S. (2004) Annu. Rev. Biochem. 73, 355-382.
10. Hay, R. T. (2005 Mol. Cell 18, 1-12.
11. Gill, G. (2005) Curr. Opin. Genet. Dev. 15, 536-541.
12. Kerscher, O., Felberbaum, R., and Hochstrasser, M. (2006) Annu. Rev. Cell Dev. Biol. 22, 159-180.
13. Bossis, G., and Melchior, F. (2006) Cell Div. 29, 1.
14. Mukhopadhyay, D., and Dasso, M. (2007) Trends Biochem. Sci. 32, 286-295.
15. Desterro, J. M., Thomson, J., and Hay, R. T. (1997) FEBS Lett 417, 297-300.
16. Johnson, E. S., and Blobel, G. (1997) J. Biol. Chem. 272, 26799-26802.
17. Rodriguez, M. S., Dargemont, C., and Hay, R. T. (2001) J. Biol. Chem. 276, 12654-12659.
18. Sampson, D. A., Wang, M., and Matunis, M. J. (2001) J. Biol. Chem. 276, 21664-21669.
19. Zhong, N., Radu, G., Ju, W. and Brown, W. T. (2005) Biochem. Biophys. Res. Commun. 338, 855-861.
20. Fatkin, D., MacRae, C., Sasaki, T., Wolff, M. R., Porcu, M., Frenneaux, M., Atherton, J., Vidaillet, H. J. Jr., Spudich, S., De Girolami, U., Seidman, J. G., Seidman, C., Muntoni, F., Muehle, G., Johnson, W., and McDonough, B. (1999) N. Engl J. Med. 341, 1715-1724.
21. Jakobs, P. M., Hanson, E. L., Crispell, K. A., Toy, W., Keegan, H., Schilling, K., Icenogle, T. B., Litt, M, and Hershberger, R. E. (2001) J. Card. Fail. 7, 249-256.
22. Xing, H., Wilkerson, D. C., Mayhew, C. N., Lubert, E. J., Skaggs, H. S., Goodson, M. L., Hong, Y., Park-Sarge, O. K., and Sarge, K. D. (2005) Science 307, 421-423.
23. Zhang, Y. and Sarge, K. D. (2008) Sumoylation regulates lamin A function and is lost in lamin A mutants associated with familial cardiomyopathies. J. Cell. Biol. 182: 35-39.

What is claimed is:

1. A method of diagnosing dilated cardiomyopathy in a subject, comprising:
(a) obtaining a biological test sample from said subject, wherein said biological test sample comprises lamin A protein;
(b) measuring the sumoylation of said lamin A protein from said biological test sample; and
(c) determining whether said lamin A protein from said biological test sample has decreased sumoylation by comparing the sumoylation obtained in step (b) with a standard sumoylation level; wherein decreased sumoylation of said lamin A protein from said biological test sample relative to said standard sumoylation level is indicative of dilated cardiomyopathy.

2. The method of claim 1, wherein said dilated cardiomyopathy is familial dilated cardiomyopathy.

3. The method of claim 1, wherein said lamin A protein from said biological test sample comprises a mutation that inhibits sumoylation.

4. The method of claim 3, wherein said mutation disrupts the sumoylation consensus sequence.

5. The method of claim 4, wherein said mutation comprises a substitution, deletion, or addition mutation within said consensus sequence.

6. The method of claim 5, wherein said mutation comprises a substitution of the glutamic acid residue at amino acid position 203 of SEQ ID NO: 1.

7. The method of claim 6, wherein said glutamic acid residue is substituted with a glycine or a lysine residue.

8. The method of claim 1, wherein said sumoylation of said lamin A protein from said biological test sample and said sumoylation of a lamin A protein from a control sample is SUMO-2 sumoylation.

9. The method of claim 1, wherein said biological test sample is a body fluid or tissue sample.

10. The method of claim 1, wherein said biological test sample is a cell extract.

11. The method of claim 10, wherein said cell extract is a lymphocyte extract.

12. The method of claim 1, wherein said subject is a mammal.

13. The method of claim 12, wherein said subject is selected from the group consisting of a human, a cat, a dog, a horse, and a cow.

14. The method of claim 13, wherein said subject is a human.

15. The method of claim 1, wherein said measuring of said sumoylation of said lamin A protein from said biological test sample comprises using an antibody that specifically binds to the sumoylated form of the lamin A protein but not to the non-sumoylated form of the lamin A protein.

16. The method of claim 1, wherein said measuring of said sumoylation of said lamin A protein from said biological test sample comprises using an antibody that specifically binds to the non-sumoylated form of the lamin A protein but not to the sumoylated form of the lamin A protein.

17. The method of claim 1, wherein said measuring of said sumoylation of said lamin A protein from said biological test sample comprises immunoprecipitation of lamin A using an anti-lamin A antibody followed by Western Blotting using an anti-SUMO-2 antibody.

18. A method for treating dilated cardiomyopathy, comprising enhancing the sumoylation of the lamin A protein in a subject in need thereof.

19. The method of claim 18, wherein said dilated cardiomyopathy is familial dilated cardiomyopathy.

20. The method of claim 18, wherein said dilated cardiomyopathy is related to decreased sumoylation of the lamin A protein of said subject.

21. The method of claim 20, wherein said lamin A protein comprises a mutation that inhibits sumoylation.

22. The method of claim 21, wherein said mutation disrupts the sumoylation consensus sequence.

23. The method of claim 22, wherein said mutation comprises a substitution, deletion, or addition mutation within said consensus sequence.

24. The method of claim 23, wherein said mutation comprises a substitution of the glutamic acid residue at amino acid position 203 of SEQ ID NO: 1.

25. The method of claim 24, wherein said glutamic acid residue is substituted with a glycine or a lysine residue.

26. The method of claim 18, wherein said sumoylation of said lamin A protein is enhanced by administering to said subject a therapeutically effective amount of an agent selected from the group consisting of a SUMO-E2 enzyme, a SUMO-E3 enzyme, and an expression construct comprising a polynucleotide sequence encoding a SUMO-E2 enzyme or a SUMO-E3 enzyme.

27. The method of claim 26, wherein said expression construct comprising a polynucleotide sequence encoding a SUMO-E2 enzyme or a SUMO-E3 enzyme is delivered via gene therapy.


SEQUENCE LISTING (4 sequences)

SEQ ID NO: 1 - protein, 664 aa, Homo sapiens
METPSQRRATRSGAQASSTPLSPTRITRLQEKEDLQELNDRLAVYIDRVRSLETENAGLRLRITESEEVVSREVSGIKAAYEAELGDARKTLDSVAKERARLQLELSKVREEFKELKARNTKKEGDLIAAQARLKDLEALLNSKEAALSTALSEKRTLEGELHDLRGQVAKLEAALGEAKKQLQDEMLRRVDAENRLQTMKEELDFQKNIYSEELRETKRRHETRLVEIDNGKQREFESRLADALQELRAQHEDQVEQYKKELEKTYSAKLDNARQSAERNSNLVGAAHEELQQSRIRIDSLSAQLSQLQKQLAAKEAKLRDLEDSLARERDTSRRLLAEKEREMAEMRARMQQQLDEYQELLDIKLALDMEIHAYRKLLEGEEERLRLSPSPTSQRSRGRASSHSSQTQGGGSVTKKRKLESTESRSSFSQHARTSGRVAVEEVDEEGKFVRLRNKSNEDQSMGNWQIKRQNGDDPLLTYRFPPKFTLKAGQVVTIWAAGAGATHSPPTDLVWKAQNTWGCGNSLRTALINSTGEEVAMRKLVRSVTVVEDDEDEDGDDLLHHHHGSHCSSSGDPAEYNLRSRTVLCGTCGQPADKASASGSGAQVGGPISSGSSASSVTVTRSYRSVGGSGGGSFGDNLVTRSYLLGNSSPRTQSPQNCSIM

SEQ ID NO: 2 - DNA, 27 bp, Artificial Sequence
  Description of Artificial Sequence Synthetic primer
ctgcagaccatgagggaggaactggac

SEQ ID NO: 3 - DNA, 27 bp, Artificial Sequence
  Description of Artificial Sequence Synthetic primer
accatgaaggagggactggacttccag

SEQ ID NO: 4 - DNA, 27 bp, Artificial Sequence
  Description of Artificial Sequence Synthetic primer
accatgaaggagaaactggacttccag